(19) United States
(12) Patent Application Publication
(10) Pub. No.: US 2022/0387381 A1
(43) Pub. Date: Dec. 8, 2022

(54) GUINEENSINE COMPOSITIONS, HERBAL COMPOSITIONS, AND METHODS OF USING THEREOF

(71) Applicant: Rocket Science Oils, LLC, San Diego, CA (US)

(72) Inventors: Steven MARK, San Diego, CA (US); Erin MARK, San Diego, CA (US); Fabio MAGRINI, La Mesa, CA (US)

(73) Assignee: Rocket Science Oils, LLC, San Diego, CA (US)

(21) Appl. No.: 17/833,736

(22) Filed: Jun. 6, 2022

Related U.S. Application Data
(60) Provisional application No. 63/208,261, filed on Jun. 8, 2021.

(51) Int. Cl. A61K 31/357; A61K 36/67; A61K 36/076; A61K 36/79; A61K 36/534; A61K 36/185; A61K 36/725

(52) U.S. Cl. CPC A61K 31/357; A61K 36/67; A61K 36/076; A61K 36/79; A61K 36/534; A61K 36/185; A61K 36/725

(57) ABSTRACT

Provided herein are compositions and methods of reducing certain psychoactive and physiological effects of tetrahydrocannabinol (THC), some of which may be undesirable, while preserving beneficial effects of cannabis in a human in need thereof. The methods described involve administration of therapeutically effective amounts of guineensine. Also, provided are compositions wherein guineensine is part of an herbal composition and is formulated for sublingual delivery.

### CROSS-REFERENCE TO RELATED APPLICATIONS

This application claims priority to U.S. Provisional Patent Application No. 63/208,261, filed Jun. 8, 2021, which is incorporated herein by reference in its entirety.

### FIELD

The present disclosure relates generally to compositions and methods for reducing psychoactive effects of tetrahydrocannabinol, and more specifically to guineensine compositions and use of such compositions.

### BACKGROUND

Cannabis sativa, commonly known as cannabis or marijuana, although it is often considered as a recreational substance, it is increasingly used for its many health benefits across many chronic conditions. While the psychoactive effects of tetrahydrocannabinol (“THC”) can be considered desirable by some, for many they can include anxiety, paranoia, panic, or a feeling of unbalance, therefore precluding its use. THC intoxication can also induce certain physiological responses such as increased heart rate and blood pressure, irregular breathing, sweating and nausea. Ways of maintaining benefits of cannabis, while attenuating or counteracting certain psychoactive and physiological effects of THC, some of which may be undesirable, is therefore desirable.

Thus, what are needed in the art are compositions that may be used to mitigate certain psychoactive and physiological effects of THC (some of which may be undesirable), while preserving beneficial effects of cannabis.

### BRIEF SUMMARY

In some aspects, provided is a method of reducing certain psychoactive and physiological effects of tetrahydrocannabinol (THC) in a human in need thereof, comprising orally administering guineensine before, after or concurrently with consumption of THC. In some embodiments, the psychoactive effects comprise anxiety, panic, paranoia, a feeling of unbalance, hallucinations, a general alteration of conscious perception, or disruption of linear memory, problems with coordination, or lowered reaction time, or any combination thereof. In some embodiments, the physiological effects comprise increased heart rate, increased blood pressure, irregular breathing, sweating, or nausea, or any combination thereof. In some embodiments, the administration of guineensine further supports and/or enhances body relaxation, feelings of well-being, stress reduction, or other medical properties of THC, or a combination thereof, in the human.

In certain aspects, provided is a a composition, comprising: a fungus, a fruit-bearing vine, a mint plant, a woody shrub, and a buckthorn, or any parts of the foregoing; and guineensine. In some embodiments, the composition is in the form of a tablet. In some variations, the tablet is formulated for sublingual delivery.

### DETAILED DESCRIPTION

The following description sets forth exemplary compositions, methods, parameters and the like. It should be recognized, however, that such description is not intended as a limitation on the scope of the present disclosure but is instead provided as a description of exemplary embodiments.

In some aspects, provided are compositions, including blends of herbs, that can counteract certain effects of THC (some of which may be undesirable), while still allowing for beneficial effects of cannabis. Administration of the compositions provided herein to a subject, such as a human who has consumed products containing THC (such as cannabis), may feel: a reduction in anxiety, paranoia, obsessive thought and panic and return to a feeling of balance; and relief from physiological symptoms associated with THC consumption, such as increased heart rate and blood pressure, irregular breathing, sweating and nausea. In some variations, the compositions provided herein provide easy dosing and bioavailability, including various formulations such as a sublingual tablet.

The compositions provided herein may be used by subjects who want to stay clear-headed and present while enjoying the other benefits of cannabis. The compositions provided may be also administered to subjects in situations of an accidental over-consumption of cannabis, bad reactions, and/or emergencies, which require the subjects to regain control quickly when life requires full focus.

Subjects with serious ailments can find relief with cannabis daily, but they may not want to feel the psychoactive effects that comes with THC. The compositions provided herein can address this problem by reducing the psychoactive effects of THC without compromising its medicinal use. The compositions provided herein allow for pro-active control of the medicinal marijuana experience.

In some aspects, provided herein are guineensine compositions that are suitable for counteracting certain effects of THC, including reducing the psychoactive effects of THC. Guineensine present in the compositions herein can serve as a cannabinoid inhibitor, reducing the amount of THC that binds to the cannabinoid receptors in the body. This can help to reduce psychoactive effects of THC. In some variations, guineensine present in the compositions herein have cannabinoid-mimetic properties that can also support and/or enhance certain desirable effects of THC, such as body relaxation and other medical properties of THC.

In some embodiments, the compositions provided herein further include an herbal blend selected to produce a calming effect in helping individuals regain a sense of balance and ease. In some embodiments, the compositions provided may further include one or more additional ingredients, such as flavoring and coloring agents (e.g., all-natural flavors and colorings), and sweeteners (e.g., zero calorie sweeteners).

Guineensine is known in the art as (2E,4E,12E)-13-(1,3-benzodioxol-5-yl)-N-(2-methylpropyl)trideca-2,4,12-trienamide, as well as by the terms Guineesine and Pipyahyine. Guineensine is generally known to induce cannabimimetic effects, such as catalepsy, in animals. See Nicolussi, Simon et al. “Guineensine Is A Novel Inhibitor Of Endocannabinoid Uptake Showing Cannabimimetic Behavioral Effects In BALB/C Mice”. Pharmacological Research, vol 80, 2014, pp. 52-65. However, certain guineensine compositions, including compositions that have a certain amount of guineensine present and optionally in combination with other herbal ingredients, were surprisingly found to counteract certain psychoactive and physiological symptoms of THC.

Guineensine can be found in long pepper (Piper longum) and black pepper (Piper nigrum). Any suitable methods known in the art to obtain a guineensine extract from these peppers may be used to provide the compositions provided herein. In some embodiments, the guineensine extracts used in the compositions herein are obtained from long pepper.

### Herbal Compositions

In certain aspects, provided herein are compositions comprising an herbal blend and guineensine. In some variations, the components of the herbal blend and guineensine may be present in varying amounts in the composition.

In some embodiments, the guineensine is present in the compositions as a long pepper extract. In some variations, the long pepper extract is present in the composition at an amount between 1 wt % and 10 wt %; or between 1 wt % and 5 wt % (where “wt % refers to weight percentage).

In some embodiments, the herbal blend comprises: a fungus, a fruit-bearing vine plant, a mint plant, a woody shrub, and/or a buckthorn, or any parts of the foregoing. In certain embodiments, the herbal blend comprises: a fungus, a fruit-bearing vine plant or its fruit, a mint plant, a woody shrub or its aromatic flowers, and/or a buckthorn or its fruit or seeds.

In some variations, the fungus is in the family of Polyporaceae. Suitable examples of the fungus may include Poria Mushroom (e.g. Poria spiritus). In certain variations, the foregoing is present in the composition at an amount between 5 wt % and 10 wt %.

In some variations, the fruit-bearing vine plant is in the family of Schisandraceae. Suitable examples of the fruit-bearing vine plant include Schizandra or Schisandra (e.g. Schisandra chinensis). In certain variations, the foregoing is present in the composition at an amount between 5 wt % and 10 wt %.

In some variations, the mint plant is in the family of Lamiaceae, Suitable examples of the mint plant include Scutellaria (e.g. Scutellaria lateriflora). In some variations, the mint plant is a hybrid of two or more plants in the Lamiaceae family. Suitable examples of a hybrid mint plant include Peppermint (e.g. Mentha piperita). In certain variations, the foregoing is present in the composition at an amount between 10 wt % and 15 wt %.

In some variations the woody shrub is in the family of Passifloraceae. Suitable examples of the woody shrub include Damiana (e.g. Turnera diffusa). In certain variations, the foregoing is present in the composition at an amount between 1 wt % and 5 wt %.

In some variations the buckthorn is in the family of Rhamnaceae. Suitable examples of buckthorn include Zizyphus (jujube) (e.g. Zizyphus jujuba, Zizyphus spinosa). In certain variations, the foregoing is present in the composition at an amount between 5 wt % and 10 wt %.

In some variations, the compositions provided comprise: guineensine, poria mushroom, schizandra, peppermint, damiana, zizyphus, and long pepper extract. In one variation the compositions provided further comprise monk fruit, vanilla flavor, peppermint flavor and bitter block, natural flavors, mannitol, microcrystalline cellulose, dicalcium phosphate, silicon dioxide, and/or magnesium stearate. In one variation, the compositions provided comprises guineensine, Poria Mushroom, Schizandra, Peppermint, Damiana, and Zizyphus (Jujube).

In certain variations, the compositions provided comprise: guineensine, Poria Spiritus Schisandra, Scutellaria Lateriflora, Turnera Diffusa, and Zizyphus Spinosa.

In some variations the compositions, the herbal blend and guineensine may be independently provided as extracts, tinctures, and/or dry herbs.

In other embodiments, the compositions provided herein may include one or more additional components or ingredients. For example, in certain variations, the compositions provided further comprise: flavoring ingredients, colorings, sweeteners, fillers or inert diluents, texturizers, emulsifiers, bulking agents, stabilizers, nutritional supplements, drying agents, buffers, thickeners, anticaking agents, cannabinoids (e.g., cannabidiol), and/or other extracts. In one variation, the compositions provided further comprise: cannabidiol, Kava or an extract obtained from Piper methysticum, lavender and/or vanilla.

In some variations, additional components or ingredients may include, for example, colorings, sweeteners, fillers or inert diluents, texturizers, emulsifiers, bulking agents, stabilizers, nutritional supplements, drying agents, buffers, thickeners, anticaking agents, cannabinoids, and/or peppers or other extracts thereof.

In some embodiments, the sweeteners are zero calorie sweeteners. In some embodiments, the flavors and colorings are all natural. In some embodiments, the compositions provided are made up of only plant-based ingredients. In some embodiments, the compositions provided are vegan.

It should be understood that the herbal blend, guineensine, and any additional components or ingredients add up to a total of 100 wt %.

The compositions provided herein may be formulated in any suitable form. In some embodiments, the compositions provided may be formulated as boluses; tablets, capsules, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

In some embodiments, the compositions are in tablet form. In some variations, the tablets are formulated for sublingual delivery.

### Methods of Use

In some aspects, provided herein are methods of reducing certain psychoactive and physiological effects of THC (including certain undesirable effects) in humans, comprising orally administering guineensine before, after or concurrently with consumption of THC.

In some embodiments, the guineensine is administered at a therapeutically effective dose. In some variations, therapeutically effective as applied to dose or amount refers to the quantity of a compound or formulation that is sufficient to result in a desired benefit after administration to a subject (e.g., a human) in need thereof.

In some variations, reducing certain psychoactive effects of THC (including certain undesirable effects) refers to a method or procedure for obtaining beneficial or desired results, which may include: (1) alleviating one or more psychoactive effects caused by or associated with consumption of THC, including anxiety, panic, paranoia, a feeling of unbalance, hallucinations, a general alteration of conscious perception, and/or disruption of linear memory (2) reducing the severity and/or duration of the aforementioned effects; and/or (3) slowing or stopping the development or progression of one or more of the aforementioned effects caused by or associated with consumption of THC.

In some variations, reducing the certain physiological effects of THC (including certain undesirable effects) refers to a method or procedure for obtaining beneficial or desired results, which may include: (1) alleviating one or more physiological effects caused by or associated with consumption of THC, including increased heart rate and blood pressure, irregular breathing, sweating, nausea, problems with coordination, and/or lowered reaction time; (2) reducing the severity and/or duration of the aforementioned effects; and/or (3) slowing or stopping the development or progression of one or more aforementioned effects caused by or associated with consumption of THC.

Any of the guineensine compositions described herein may be used in such methods. In some embodiments, the guineensine compositions are used to counteract the certain effects of THC (including certain undesirable effects). For example, in one variation, the composition is used before, after or concurrently with the medicinal or recreation use of cannabis or THC to maintain control of the cannabis experience. The composition is administered to counteract THC and certain psychoactive and physiological effects of THC (including certain undesirable effects) while still allowing for certain benefits of cannabis. The compositions herein may take effects within a few minutes. In one variation, the subject administered the guineensine composition may experience one or more effects, such as a reduction in anxiety and panic and return to a feeling of balance; and relief from physiological effects common with THC consumption such as increased heart rate and blood pressure, irregular breathing, sweating and nausea. In one variation, the subject may feel more clear-headed and present, while still obtaining the desired benefits of cannabis.

In other embodiments, the guineensine compositions provided may be administered to reduce certain psychoactive effects from the recreational use of cannabis or THC. For example, the subject can enjoy the experience of cannabis or THC by consuming the compositions herein, which gives the subject the ability to mitigate and/or counteract certain effects of THC (including certain undesirable effects) when he/she chooses. The composition is administered so that the subject regains more control and/or the ability to focus. In some variations, the subject has had a negative response to THC or cannabis or the user is new to the use of cannabis. In some variations, the subject has experienced accidental over-consumption, bad reactions or emergencies.

In some embodiments, the guineensine compositions are used for medicinal relief without feeling certain psychoactive effects (including certain undesirable effects). For example, subjects with serious ailments may find relief with the use of cannabis but may not want to feel certain psychoactive effects (including certain undesirable effects) that comes with THC. The guineensine compositions may be administered to reduce the psychoactive effects of THC without compromising its medicinal benefits. In some variations, the guineensine compositions allow for pro-active control of the medicinal marijuana experience. In some embodiments, the guineensine compositions can also yield a relaxing and calming effect on the subject.

In some embodiments, the guineensine compositions are used to support or enhance the desirable effects of THC. For example, the compositions may induce cannabinoid-mimetic properties such as body relaxation, feelings of well-being, stress reduction, and other medical properties of THC.

### Modes of Administration

In some variations, the guineensine is formulated for sublingual delivery.

Other suitable modes of administration may be include, for example, oral, parenteral (including intramuscular, subcutaneous, intradermal, intravascular, intravenous, intraarterial, intraperitoneal, intramedullary, intrathecal and topical), intracavitary, and topical (including dermal/epicutaneous, transdermal, mucosal, transmucosal, intranasal (e.g., by nasal spray or drop), pulmonary (e.g., by oral or nasal inhalation), ocular (e.g., by eye drop), buccal, sublingual, rectal (e.g., by suppository) and vaginal (e.g., by suppository)).

In some variations, the compositions comprising guineensine are formulated as a tablet. In certain variations, the compositions comprising guineensine are formulated as a tablet for sublingual delivery.

In certain variations, the compositions are administered to a subject in need thereof as a sublingual tablet (e.g., placed under the tongue of the subject until dissolved). In certain variations, a second sublingual tablet of the composition may be administered to the subject, provided that the compositions administered do not exceed six sublingual tablets in a 24-hour period.

In others variations, the compositions comprising guineensine are formulated in liquid form.

In yet other variations the compositions, the herbal blend and guineensine present in the compositions (including, for example, as a tablet or liquid formulation) may be independently provided as extracts, tinctures, and/or dry herbs (as the case may be).

What is claimed is:

1. A method of selectively reducing psychoactive and physiological effects of tetrahydrocannabinol (THC) in a human in need thereof, comprising orally administering guineensine before, after or concurrently with consumption of THC.

2. The method of claim 1, wherein the psychoactive effects comprise anxiety, panic, paranoia, a feeling of unbalance, hallucinations, a general alteration of conscious perception, or disruption of linear memory, problems with coordination, or lowered reaction time, or any combination thereof.

3. The method of claim 1, wherein the physiological effects comprise increased heart rate, increased blood pressure, irregular breathing, sweating, or nausea, or any combination thereof.

4. The method of claim 1, wherein the administration of guineensine further supports and/or enhances body relaxation, feelings of well-being, stress reduction, or other medical properties of THC, or a combination thereof, in the human.

5. The method of claim 1, wherein the guineensine is provided in a long pepper extract.

6. The method of claim 1, wherein the guineensine is provided as part of an herbal composition.

7. The method of claim 6, wherein the herbal composition further comprises: colorings, sweeteners, fillers or inert diluents, texturizers, emulsifiers, bulking agents, stabilizers, nutritional supplements, drying agents, buffers, thickeners, anticaking agents, cannabinoids, and/or other extracts.

8. The method of claim 6, wherein the herbal composition further comprises: Poria Mushroom, Schizandra, Peppermint, Damiana, and/or Zizyphus (Jujube).

9. The method of claim 6, wherein the herbal composition further comprises a monk fruit sweetener, flavorant(s), mannitol, microcrystalline cellulose, dicalcium phosphate, silicon dioxide, and/or magnesium stearate.

10. The method of claim 1, wherein the guineensine is administered sublingually to the human.

11. The method of claim 1, wherein the guineensine is formulated as a tablet.

12. A composition, comprising:
a fungus, a fruit-bearing vine, a mint plant, a woody shrub, and a buckthorn, or any parts of the foregoing; and
guineensine.

13. The composition of claim 12, wherein:
the fungus is Poria Mushroom;
the fruit-bearing vine is Schisandra;
the mint plant is Peppermint (Mentha piperita) or Scutellaria lateriflora;
the woody shrub is Damiana (Turnera diffusa); and
the buckthorn is Zizyphus (Jujube, Ziziphus spinosa).

14. The composition of claim 12, wherein the guineensine is provided in a long pepper extract.

15. The composition of claim 14, wherein the long pepper extract is present in an amount between 1 wt % and 10 wt % of the total composition.

16. The composition of claim 12, wherein the composition comprises: guineensine, poria mushroom, schizandra, peppermint, damiana, zizyphus (jujube), and long pepper extract.

17. The composition of claim 12, wherein the composition further comprises: colorings, sweeteners, fillers or inert diluents, texturizers, emulsifiers, bulking agents, stabilizers, nutritional supplements, drying agents, buffers, thickeners, anticaking agents, cannabinoids, and/or peppers.

18. The composition of claim 12, wherein the composition further comprises: a monk fruit sweetener, vanilla flavor, peppermint flavor and bitter block, natural flavors, mannitol, microcrystalline cellulose, dicalcium phosphate, silicon dioxide, and/or magnesium stearate.

19. The composition of claim 12, wherein the composition is in the form of a tablet.

20. The composition of claim 19, wherein the tablet is formulated for sublingual delivery.
